(19) United States
(12) Patent Application Publication
(10) Pub. No.: US 2004/0143099 A1
(43) Pub. Date: Jul. 22, 2004

(54) Tissue factor antagonist and blood glucose regulator compositions

(76) Inventors: Lars Christian Petersen, Horsholm (DK); Jakob Michael Back, Rungsted Kyst (DK); Christian Meyer, Humlebaek (DK)

Correspondence Address: Reza Green, Esq., Novo Nordisk Pharmaceuticals, Inc., 100 College Road West, Princeton, NJ, 08540, US

(21) Appl. No.: 10/701,294

(22) Filed: Nov. 4, 2003

Related U.S. Application Data
(60) Provisional application No. 60/434,904, filed on Dec. 20, 2002.

(30) Foreign Application Priority Data
Nov. 6, 2002 (DK) PA 2002 01710

(51) Int. Cl. A61K 39/395; C07K 16/28

(52) U.S. Cl. 530/383; 514/12; 530/388.22

(57) ABSTRACT

Compositions comprising a tissue factor antagonist and a blood glucose regulator, methods of promoting, inducing, and/or enhancing a physiological response in a subject comprising administering such a composition or combination of tissue factor antagonist and blood glucose regulator, such as in the treatment or prevention of a thrombotic or coagulopathic disease, respiratory disease, or inflammatory disease.

### CROSS-REFERENCE TO RELATED APPLICATIONS

This application claims priority under 35 U.S.C. 119 of Danish application no. PA 2002 01710 filed Nov. 6, 2002 and U.S. application No. 60/434,904 filed Dec. 20, 2002, the contents of which are fully incorporated herein by reference.

### FIELD OF THE INVENTION

The invention described herein relates to compositions comprising a tissue factor antagonist and a blood glucose regulator. The invention also relates to the use of combinations of TF antagonists and blood glucose regulators in the prevention and/or treatment of thrombotic or coagulopathic related diseases, respiratory diseases, and inflammatory diseases.

### BACKGROUND OF THE INVENTION

Tissue Factor (TF) is a cellular transmembrane receptor for plasma coagulation factor VIIa and formation of TF/FVIIa complexes on the cell surface triggers the coagulation cascade in vivo, The TF/FVIIa complex efficiently activates coagulation factors IX and X. The resultant protease factor Xa (Xa), activates prothrombin to thrombin, which in turn converts fibrinogen into a fibrin matrix.

Normally, TF is constitutively expressed on the surface of many extravascular cell types that are not in contact with the blood, such as fibroblasts, pericytes, smooth muscle cells and epithelial cells, but not on the surface of cells that come in contact with blood, such as endothelial cells and monocytes. However, TF is also expressed in various pathophysiological conditions where it is believed to be involved in progression of disease states within cancer, inflammation, atherosclerosis, and ischemia/reperfusion. Thus tissue factor is now recognized as a target for therapeutic intervention in conditions associated with increased expression.

Factor VIIa (FVIIa) is a two-chain, 50 kilodalton (kDa) vitamin-K dependent, plasma serine protease which participates in the complex regulation of in vivo hemostasis. FVIIa is generated from proteolysis of a single peptide bond from its single chain zymogen, Factor VII (FVII), which is present at approximately 0.5 pg/ml in plasma. The zymogen is catalytically inactive. The conversion of zymogen FVII into the activated two-chain molecule occurs by cleavage of an internal peptide bond. In the presence of calcium ions, FVIIa binds with high affinity to exposed TF, which acts as a cofactor for FVIIa, enhancing the proteolytic activation of its substrates FVII, Factor IX, and FX.

In addition to its established role as an initiator of the coagulation process, TF was recently shown to function as a mediator of intracellular activities either by interactions of the cytoplasmic domain of TF with the cytoskeleton or by supporting the VIIa-protease dependent signaling. Such activities may be responsible, at least partly, for the implicated role of TF in tumor development, metastasis, and angiogenesis. Cellular exposure of TF activity is advantageous in a crisis of vascular damage but may be fatal when exposure is sustained as it is in these various diseased states. Thus, it is critical to regulate the expression of TF activity in maintaining the health.

Inactivated FVII (FVIIai) is FVIIa modified in such a way that it is catalytically inactive. FVIIa is thus not able to catalyze the conversion of FX to FXa, but still able to bind to TF in competition with active endogenous FVIIa and thereby inhibit the TF activity.

International patent applications WO 92/15686, WO 94/27631, WO 96/12800, WO 97/47651 relates to FVIIai and the uses thereof. International patent applications WO 90/03390, WO 95/00541, WO 96/18653, and European Patent EP 500800 describe peptides derived from FVIIa having TF/FVIIa antagonist activity. International patent application WO 01121661 relates to bivalent inhibitors of FVII and FXa.

It is often necessary to selectively block the coagulation cascade in a patient. Anticoagulants such as heparin, coumarin, derivatives of coumarin, indandione derivatives, or other agents may be used. For example, during kidney dialysis, or to treat deep vein thrombosis, disseminated intravascular coagulation (DIC), and a host of other medical disorders.

Treatment with heparin and other anticoagulants may, however, have undesirable side effects. Available anticoagulants generally act throughout the body, rather than acting specifically at a clot site. Heparin, for example, may cause heavy bleeding. Because heparin acts as a cofactor for antithrombin III (AT III), and AT III is rapidly depleted in DIC treatment, it is often difficult to maintain the proper heparin dosage, necessitating continuous monitoring of AT III and heparin levels. Heparin is also ineffective if AT III depletion is extreme. Further, prolonged use of heparin may also increase platelet aggregation and reduce platelet count, and has been implicated in the development of osteoporosis. Indandione derivatives may also have toxic side effects. In addition to the anticoagulants briefly described above, several naturally occurring proteins have been found to have anticoagulant activity, such as, for example, anticoagulant proteins isolated anticoagulant proteins from bovine aorta and human umbilical vein arteries and human placenta-derived anticoagulant proteins.

Inhibitors of tissue factor may act as antagonists for tissue factor-mediated induction of coagulation, thus blocking the production of thrombin and the subsequent deposition of fibrin. As such, TF antagonists may be useful for inhibiting tissue factor activity resulting in, for example, the inhibition of blood coagulation, thrombosis, pulmonary embolism, stroke, disseminated intravascular coagulation (DIC), platelet deposition, fibrin deposition in lungs and kidneys associated with gram-negative endotoxemia, myocardial infarction, and inflammatory responses including acute lung injury, acute respiratory distress syndrome, and systemic inflammatory response syndrome.

Currently, no TF antagonists have been developed and marketed for therapeutic use in humans. Known therapeutic strategies include monoclonal antibodies, catalytically impaired FVIIa mutants and chemically inactivated FVIIa.

Therapeutic use of mouse Mabs against TF is known from U.S. Pat. Nos. 6,001,978 and 5,223.427.

International Application No. WO 99151743 relates to human/mouse chimera monoclonal antibodies directed against human TF.

European patent application No. 83391 1 relates to CDR-grafted antibodies against human TF.

Presta L. et al., Thrombosis and Haemostasis, Vol. 85 (3) pp. 379-389 (2001) relates to humanized antibody against TF.

Critical illness polyneuropathy (CIP) is a syndrome that was first extensively described in the early 1980s, mainly in patients with failure to wean from mechanical ventilation. The syndrome is further characterized by limb muscle weakness, usually more pronounced distally than proximally, and is often accompanied by atrophy. The facial musculature is often strikingly spared, Reduced or absent deep-tendon reflexes and loss of peripheral sensation to light touch and pin prick often accompany the syndrome. Involvement of the phrenic nerve has been shown to further contribute to delayed weaning from the ventilator in many patients. The electrophysiologic studies are consistent with a predominantly motor and, often to a lesser extent, sensory axonal polyneuropathy. The incidence of CIP is high, with often more than 50% of patients in major medical and surgical critical care units suffering from the syndrome. The systemic inflammatory response syndrome (SIRS) is strongly associated with CIP and, among the multiorgan failure often seen in SIRS, CIP is thought to represent a neurologic manifestation of SIRS. CIP occurs in about 70% of patients who have the systemic inflammatory response syndrome (SIRS). The neurologic effects of SIRS are thought to be mediated by released mediators like cytokines and free radicals, affecting the microcirculation of the central and peripheral nervous system. Examination of the peripheral nervous system is often unreliable, and the only way to establish a definitive diagnosis is by performing electrophysiologic studies. Morbidity and mortality rates are high. If the underlying problem causing sepsis and/or SIRS can be treated successfully, full recovery from CIP can occur. This recovery often occurs in a matter of weeks in milder cases and in months in more severe cases. Knowledge of CIP is essential for intensivists and other specialists who care for critically ill patients. However, clinical signs are often absent and it remains an occult problem in many ICUs worldwide. Nonetheless, it is an important clinical entity as it is a frequent cause of difficulty to wean patients from the ventilator and it leads to problems with rehabilitation after the acute illness has been treated and cured.

If the underlying condition (e.g., sepsis or SIRS) can be successfully treated, recovery from and/or prevention of CIP can be expected. This will occur in a matter of weeks in mild cases and in months in more severe cases.

The pathophysiology of this type of neuropathy remains unknown. It has been speculated to be directly related to inflammatory diseases or disorders such as, e.g., sepsis and its mediators. Indeed, cytokines released in sepsis have histamine-like properties which may increase microvascular permeability and induce disturbed microcirculation.

International Publication No. WO 01185256 relates to a medicament for critically ill patients and a method of treatment; the medicament is a blood glucose regulator.

There is still a need in the art for improved compositions and treatments having anticoagulant and anti-inflammatory activity which can be administered at relatively low doses and do not produce the undesirable side effects associated with traditional anticoagulant compositions. The present invention fulfils this need by providing anticoagulants that act specifically at sites of injury or TF exposure, and further provides other related advantages. Furthermore the present invention provides compositions, which act to inhibit the cellular functions of TF, which is implicated in Respiratory and Inflammatory conditions like sepsis, inflammation, atherosclerosis, restenosis, or cancer.

### SUMMARY OF THE INVENTION

One object of the present invention is to provide compositions, which can effectively be used in the treatment or prophylaxis of Thrombotic or Coagulopathic related disease, Respiratory diseases, and inflammatory diseases.

Other objects of the present invention will become apparent upon reading the present description.

In a first aspect the invention provides a pharmaceutical composition comprising a TF antagonist and a blood glucose regulator.

In a second aspect, the invention provides the use of a TF antagonist in combination with a blood glucose regulator for the manufacture of a medicament for treating Thrombotic and Coagulopathic related diseases or disorders, Respiratory diseases or disorders, and Inflammatory diseases or disorders.

In one embodiment thereof, the Thrombotic and Coagulopathic related diseases or disorders, Respiratory diseases or disorders, and Inflammatory diseases or disorders include deep venous thrombosis, arterial thrombosis, post surgical thrombosis, coronary artery bypass graft (CABG), percutaneous transdermal coronary angioplastry (PTCA), stroke, tumor metastasis, inflammation, septic chock, hypotension, acute lung injury (ALI), Acute Respiratory Distress Syndrome (ARDS), pulmonary embolism, disseminated intravascular coagulation (DIC), sepsis, systemic inflammatory response syndrome (SIRS), vascular restenosis, platelet deposition, myocardial infarction, angiogenesis, or the prophylactic treatment of mammals with atherosclerotic vessels at risk for thrombosis; asthma, bronchitis, idiopathic pulmonary fibrosis, pneumonia, pulmonary edema, pulmonary obstructive disease, endotoxin induced lung damage, non cell lung cancer; inflammatory bowel disease, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondylarthropathris, ankylosing spondylitis, Reiter's syndrome, psoriatic arthropathy, enterapathric spondylitis, juvenile arthropathy or juvenile ankylosing spondylitis, reactive arthropathy, infectious or post-infectious arthritis, gonoccocal arthritis, tuberculous arthritis, viral arthritis, fungal arthritis, syphilitic arthritis, Lyme disease, arthritis associated with vasculitic syndromes, polyarteritis nodosa, hypersensitivity vasculitis, Luegenec's granulomatosis, polymyalgin rheumatica, joint cell arteritis, calcium crystal deposition arthropathris, pseudo gout, non-articular rheumatism, bursitis, tenosynomitis, epicondylitis (tennis elbow), carpal tunnel syndrome, repetitive use injury (typing), miscellaneous forms of arthritis, neuropathic joint disease (charco and joint), hemarthrosis (hemarthrosic), Henoch Schonlein Purpura, hypertrophic osteoarthropathy, multicentric reticulohistiocytosis, arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, relapsing, and critical illness polyneuropathy (CIP) and/or multiple organ failure resulting from any of the preceding pathologic processes.

In one embodiment, the diseases or disorders are Respiratory disease and Inflammatory disease. In one embodiment, Respiratory disease and Inflammatory disease include lower respiratory diseases such as systemic inflammatory response syndrome, asthma, bronchitis, acute lung injury, acute respiratory distress syndrome, idiopathic pulmonary fibrosis, pneumonia, pulmonary edema, pulmonary obstructive disease, endotoxin induced lung damage, non cell lung cancer; inflammatory bowel disease, sepsis, septic shock, acute respiratory distress syndrome, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondylarthropathris, ankylosing spondylitis, Reiter's syndrome, psoriatic arthropathy, enterapathric spondylitis, juvenile arthropathy or juvenile ankylosing spondylitis, reactive arthropathy, infectious or post-infectious arthritis, gonoccocal arthritis, tuberculous arthritis, viral arthritis, fungal arthritis, syphilitic arthritis, Lyme disease, arthritis associated with vasculitic syndromes, polyarteritis nodosa, hypersensitivity vasculitis, Luegenec's granulomatosis, polymyalgin rheumatica, joint cell arteritis, calcium crystal deposition arthropathris, pseudo gout, non-articular rheumatism, bursitis, tenosynomitis, epicondylitis (tennis elbow), carpal tunnel syndrome, repetitive use injury (e.g., from typing), miscellaneous forms of arthritis, neuropathic joint disease (charco and joint), hemarthrosis (hemarthrosic), Henoch-Schonlein Purpura, hypertrophic osteoarthropathy, multicentric reticulohistiocytosis. arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, relapsing, and critical illness polyneuropathy (CIP) and/or multiple organ failure resulting from any of the preceding pathologic processes In another embodiment, the diseases or disorders are Thrombotic or Coagulopatic related diseases or disorders.

In one embodiment, Thrombotic or Coagulopatic related disease include vascular diseases and inflammatory responses such as deep venous thrombosis, arterial thrombosis, post surgical thrombosis, coronary artery bypass graft (CABG), percutaneous transdermal coronary angioplastry (PTCA), stroke, tumor metastasis, inflammation, septic chock, hypotension, acute lung injury (ALI), Acute Respiratory Distress Syndrome (ARDS), pulmonary embolism, disseminated intravascular coagulation (DIC), sepsis, systemic inflammatory response syndrome (SIRS), vascular restenosis, platelet deposition, myocardial infarction, angiogenesis, or the treatment of mammals with atherosclerotic vessels at risk for thrombosis, and critical illness polyneuropathy (CIP) and/or multiple organ failure resulting from any of the preceding pathologic processes

In a preferred embodiment, the disease or disorder is one or more of systemic inflammatory response syndrome, acute lung injury, acute respiratory distress syndrome, disseminated intravascular coagulation, sepsis, or critical illness polyneuropathy (CIP) and/or multiple organ failure resulting from any of the preceding pathologic processes.

In another embodiment, the medicament is formulated for intravenous administration, preferably injection or infusion, in particular injection.

In one embodiment, the medicament is formulated in single-unit dosage form; in another it is formulated in the form of a first unit dosage form comprising a preparation of a TF antagonist and a second unit dosage form comprising a preparation of a blood glucose regulator.

In a further aspect, the invention provides a method for treating Thrombotic or Coagulopathic related disease, Respiratory disease and Inflammatory disease in a subject, the method comprising administering to a subject in need thereof a first amount of a preparation of a TF antagonist and a second amount of a preparation of a blood glucose regulator, wherein the first and second amount together are effective to treat Thrombotic or Coagulopathic related disease, Respiratory disease and Inflammatory disease.

In one embodiment the TF antagonist and the blood glucose regulator are present in a ratio by mass of between about 100:1 and about 1:100 (w/w TF antagonist:blood glucose regulator).

In another embodiment, the pharmaceutical composition is formulated for intravenous administration, preferably injection or infusion, in particular injection. In one embodiment, the composition contains at least one pharmaceutical acceptable excipients or carrier.

In one embodiment of the invention, the TF antagonist and the blood glucose regulator are administered simultaneously. In another embodiment, the TF antagonist and the blood glucose regulator are administered sequentially.

In one embodiment of the present invention, the pharmaceutical composition is in single-dosage form and consists essentially of a preparation of a TF antagonist and a preparation of a blood glucose regulator, and one or more of the components selected from the list of pharmaceutical acceptable excipients or carriers, stabilizers, detergents, neutral salts, antioxidants, preservatives, and protease inhibitors.

In another embodiment, the pharmaceutical composition is in the form of a first-unit dosage form and a second-unit dosage form, where the first-unit dosage form consists essentially of a preparation of a TF antagonist and one or more of the components selected from the list of pharmaceutical acceptable excipients or carriers, stabilizers, detergents, neutral salts, antioxidants, preservatives, and protease inhibitors; and the second-unit dosage form consists essentially of a preparation of a blood glucose regulator and one or more of the components selected from the list of pharmaceutical acceptable excipients or carriers, stabilizers, detergents, neutral salts, antioxidants, preservatives, and protease inhibitors.

In one embodiment of the present invention the TF antagonist is factor VII polypeptides chemically inactivated in the active site. In one embodiment of the present invention the TF antagonist is an antibody against TF. In one embodiment the antibody is a monoclonal antibody.

In one embodiment the antibody is a fully human monoclonal antibody. In one embodiment the antibody is a humanized monoclonal antibody such as a mouse/human chimera antibody. In one embodiment the antibody is an antibody against human TF. In one embodiment, the TF antagonist is selected from a list of a Fab fragment, a monovalent fragment consisting of the VL, VH, CL and CH1 domains; a F(ab)2 or F(ab′)2 fragment; a bivalent fragment comprising two Fab fragments linked by a disulfide bridge at the hinge region; a Fd fragment consisting of the VH and CH1 domains; a Fv fragment consisting of the VL and VH domains of a single arm of an antibody, a dAb fragment; an isolated complementarity determining region (CDR); and single chain Fv (scFv).

In one embodiment, the TF antagonist is selected from a list consisting of human and bovine Factor VII, wherein the active site residue Ser344 is modified, replaced with Gly, Met, Thr, or more preferably, Ala. Such substitution can be made separately or in combination with substitution(s) at other sites in the catalytic triad, which includes His193 and Asp242.

In one embodiment of the invention, the TF antagonist is human factor VII, which has an amino acid substitution of the lysine corresponding to position 341 of SEQ ID NO: 1.

In one embodiment of the invention, he TF antagonist is human factor VII, which has an amino acid substitution of the serine corresponding to position 344 of SEQ ID NO: 1.

In one embodiment of the invention, the TF antagonist is human factor VII, which has an amino acid substitution of the aspartic acid corresponding to position 242 of SEQ ID NO: 1.

In one embodiment of the invention, the TF antagonist is human factor VII, which has an amino acid substitution of the histidine corresponding to position 193 of SEQ ID NO: 1.

In one embodiment, the TF antagonist is a factor VII polypeptide selected from the list consisting of FVII-(K341A), FVII-(S344A), FVII-(D242A) and FVII-(H193A).

In one embodiment, the TF antagonist is a fragment of FVII.

In one embodiment, the TF antagonist is a factor VII polypeptide inactivated in the active site by reaction with a reagent selected from the list of peptide chloromethylketones or peptidyl cloromethanes; azapeptides; acylating agents such as various guanidinobenzoate derivatives and 3-alkoxy-4-chloroisocoumarins; sulphonyl fluorides such as 5 phenylmethylsulphonylfluoride (PMSF); diisopropylfluorophosphate (DFP); tosylpropylchloromethyl ketone (TPCK); tosylysylchloromethyl ketone (TLCK); nitrophenylsulphonates; heterocyclic protease inhibitors such as isocoumarines, and coumarins.

In one embodiment, the TF antagonist is a factor VII polypeptide inactivated in the active site by reaction with a reagent selected from the list of Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethyl ketone, D-Phe-Pro-Arg chloromethyl ketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone and Dansyl-D-Glu-Gly-Arg chloromethylketone.

In one embodiment, the blood glucose regulator are selected from the list of insulin, active insulin derivatives, insulin analogues, compounds that stimulate signal transduction mediated by an insulin receptor type tyrosine kinase in a cell, certain protein-tyrosine phosphatases (PTPs), other type II antidiabetica, and other biologically active substances having insulin releasing action. In one embodiment, the blood glucose regulator is porcine insulin, human insulin, or zinc salts thereof, or protamine salts thereof. In one embodiment, the blood glucose regulator is insulin aspart (i.e., Asp628 human insulin), insulin lispro (i.e., LysB28, ProB29 human insulin), insulin glagin (i.e., GlyA21, ArgB31, ArgB32 human insulin), or insulin detemir (i.e., des-ThrB30 human insulin gamma-LysB29 tetradecanoyl). In one embodiment, the patient is a non-diabetic patient.

### DETAILED DESCRIPTION OF THIS INVENTION

Tissue Factor Antagonists

The terms “TF antagonist” or “TF antagonists”, as used herein is intended to mean any compound binding directly to TF and inhibiting the activation, or conversion, of factor X to factor Xa. In practicing the present invention, any such compound binding directly to tissue factor and inhibiting conversion of factor X to factor Xa may be used. This includes, without limitation, factor VII polypeptides having substantially reduced catalytic activity, inhibitory antibodies against TF (“anti-TF antibodies”), as well as fragments thereof. Anti-TF antibodies include, without limitation, antibodies preventing or inhibiting binding of FVII to tissue factor and antibodies preventing or inhibiting binding of FIX or FX to TF/FVIIa complex. The TF antagonists bind to tissue factor with high affinity and specificity but do not initiate blood coagulation.

In one embodiment of the invention, TF antagonists encompass those that exhibit at least about 10%, at least about 20%, at least about 25%, at least about 30%, at least about 40%, at least about 50%, at least about 60%, at least about 70%, at least about 75%, at least about 80%, at least about 90%, at least about 100%, at least about 110%, at least about 120%, or at least about 130% of the specific TF-binding affinity of wild-type factor VIIa, when tested in one or more of the TF binding assays as described in the present specification. In a preferred embodiment, the TF antagonists exhibit at least about 75% of the binding affinity of wild-type factor VIIa. The term “TF binding activity” as used herein means the ability of an FVIIa polypeptide or TF antagonist to inhibit the binding of recombinant human 125I-FVIIa to cell surface human TF. The TF binding activity may be measured as described in Assay 3 (of the present specification).

In another embodiment, TF antagonists encompass those that exhibit less than about 50%, preferably less than about 25%, more preferably less than about 10%, or 5%, or 3%, or 2%, most preferably less than about 1% of the specific activity of wild-type factor VIIa, when tested in one or more of a clotting assay, or proteolysis assay as described in the present specification.

The TF antagonists for use in the present invention include, without limitation, immunoglobulin molecules and fragments thereof that have the ability to specifically bind to an antigen (i.e., TF) such as (i) a Fab fragment, a monovalent fragment consisting of the VL, VH, CL and CH I domains; (ii) F(ab)2 and F(ab′)2 fragments; (iii) a Fd fragment consisting of the VH and CH1 domains; (iv) a Fv fragment consisting of the VL and VH domains of a single arm of an antibody, (v) a dAb fragment; (vi) an isolated complementarity determining region (CDR); (vii) single chain Fv (scFv); and (viii) diabodies. Included are also antibodies having variable and constant regions derived from human germ line immunoglobulin sequences; human antibodies including amino acid residues not encoded by human germ line immunoglobulin sequences (e.g., mutations introduced by random or site-specific mutagenesis in vitro or by somatic mutation in vivo), for example in the CDRs and in particular CDR3; antibodies in which CDR sequences derived from the germ line of another mammalian species, such as a mouse, have been grafted onto human framework sequences, e.g. the so-called humanized antibodies or human/mouse chimera antibodies.

The TF antagonists for use in the present invention also encompass, without limitation, factor VII polypeptides that has substantially reduced ability to catalyze the conversion of factor X to factor Xa (“inactivated” factor VII polypeptides).

The terms “FVII polypeptide” or “FVII polypeptides” as used herein include, without limitation, native Factor VII, as well as factor VII-related polypeptides that have either been chemically modified relative to human factor VII and/or contain one or more amino acid sequence alterations relative to native Factor VII (i.e., Factor VII variants), and/or contain truncated amino acid sequences relative to native Factor VII (i.e., Factor VII fragments).

It further encompasses polypeptides with a slightly modified amino acid sequence, for instance, polypeptides having a modified N-terminal end including N-terminal amino acid deletions or additions, and/or polypeptides that have been chemically modified relative to human factor VIIa. Such factor VII-related polypeptides may exhibit different properties relative to native Factor VII, including stability, phospholipid binding, altered specific proteolytic activity, and the like. Factor VII-related polypeptides also include proteolytically inactive variants of Factor VII.

The terms “variant” or “variants”, as used herein, is intended to designate human Factor VII having the sequence of SEQ ID NO: 1, wherein one or more amino acids of the parent protein have been substituted by another amino acid and/or wherein one or more amino acids of the parent protein have been deleted and/or wherein one or more amino acids have been inserted in the protein and/or wherein one or more amino acids have been added to the parent protein.

The terms “Factor VII” or “FVII” are intended to include Factor VII polypeptides in their uncleaved (zymogen) form as well as those that have been proteolytically processed to yield their respective bioactive forms, which may be designated “factor VIIa polypeptides” (or “activated factor VII polypeptides”). Typically, FVII is cleaved between residues 152 and 153 to yield FVIIa. The term “factor VII polypeptide” is also intended to encompass, without limitation, polypeptides having the amino acid sequence 1-406 of wild-type human Factor VII (as disclosed in U.S. Pat. No. 4,784,950), as well as wild-type Factor VII derived from other species, such as, e.g., bovine, porcine, canine, murine, and salmon Factor VII. It further encompasses natural allelic variations of Factor VII that may exist and occur from one individual to another. Also, degree and location of glycosylation or other post-translation modifications may vary depending on the chosen host cells and the nature of the host cellular environment. In one series of embodiments, factor VII polypeptides include polypeptides that exhibit at least about 70%, preferably at least about 80%, more preferably at least about 90%, and most preferable at least about 95%, of identity with the amino acid sequence of wild-type factor VII as disclosed in U.S. Pat. No. 4,784,950.

Non-limiting examples of factor VII variants having substantially reduced or modified biological activity relative to wild-type factor VII include R152E-FVIIa (Wildgoose et al., Biochem 29:3413-3420, 1990), S344A-FVIIa (Kazama et al., J. Biol. Chem. 270:66-72, 1995). FFR-FVIIa (Holst et al., Eur. J. Vasc. Endovasc. 5urg. 15:515-520. 1998). and factor VIIa lacking the Gla domain, (Nicolaisen et al., FEBS Letts. 317:245-249, 1993). Non-limiting examples also include human FVIIa, which has an amino acid substitution of the lysine corresponding to position 341 of SEQ ID NO: 1; human FVIIa, which has an amino acid substitution of the serine corresponding to position 344 of SEQ ID NO: 1; human FVIIa, which has an amino acid substitution of the aspartic acid corresponding to position 242 of SEQ ID NO: 1; human FVIIa, which has an amino acid substitution of the histidine corresponding to position 193 of SEQ ID NO: 1; FVII-(K341A); FVII-(S344A); FVIL(D242A); and FVII-(H193A). Non-limiting examples of chemically modified factor VII polypeptides and sequence variants are described, e.g., in U.S. Pat. No. 5,997,864.

Non-limiting examples of FVII-derived peptides having TF/FVIIa antagonist activity are described in International patent applications WO 90/03390, WO 95/00541, WO 96/18653, and European Patent EP 500800.

The catalytic activity of Factor VIIa can be inhibited by chemical derivatization of the catalytic centre, or triad. Derivatization may be accomplished by reacting Factor VII with an irreversible inhibitor such as an organophosphor compound, a sulfonyl fluoride, a peptide halomethyl ketone or an azapeptide, or by acylation, for example, peptide chloromethylketones or peptidyl cloromethanes; azapeptides; acylating agents such as various guanidinobenzoate derivatives and 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosylysylchloromethyl ketone (TLCK); nitrophenylsulphonates; heterocyclic protease inhibitors such as isocoumarines, and coumarins.

Preferred peptide halomethyl ketones include Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethyl ketone, ,Dansyl-D-Phe-Pro-Arg chloromethyl ketone, DansyCPhe-Pro-Arg chloromethyl ketone, DansyCD-Phe-Pro-Arg chloromethylketone, Dansyl-L-Glu-Gly-Arg chlorornethylketone and Dansyl-D-Glu-Gly-Arg chloromethylketone.

In preferred embodiments amino acid substitutions are made in the amino acid sequence of the Factor VII catalytic triad, defined herein as the regions which contain the amino acids which contribute to the Factor VIIa catalytic site. The substitutions, insertions or deletions in the catalytic triad are generally at or adjacent to the amino acids which form the catalytic site. In the human and bovine Factor VII proteins, the amino acids which form a catalytic “triad” are Ser344, Asp242, and His193 (subscript numbering indicating position in SEQ ID NO:1). The catalytic sites in Factor VII from other mammalian species may be determined using presently available techniques including, among others, protein isolation and amino acid sequence analysis. Catalytic sites may also be determined by aligning a sequence with the sequence of other serine proteases, particularly chymotrypsin, whose active site has been previously determined (Sigler et al., J. Mol. Biol., 35:143-164 (1968). incorporated herein by reference), and therefrom determining from said alignment the analogous active site residues. The amino acid substitutions, insertions, or deletions are made so as to prevent or otherwise inhibit activation by the Factor VIIa of Factors X and/or IX. The Factor VII so modified should, however, also retain the ability to compete with authentic Factor VII and/or Factor VIIa for binding to tissue factor in the coagulation cascade. Such competition may readily be determined by means of, e.g., a clotting assay as described herein, or a competition binding assay using, e.g., a cell line having cell-surface tissue factor, such as the human bladder carcinoma cell line J82 (Sakai et al. J. Biol. Chem. 264: 9980-9988 (1989)).

The amino acids which form the catalytic site in Factor VII, such as Ser344, Asp242, and His193 in human and bovine Factor VII, may either be substituted or deleted. It is preferred to change only a single amino acid, thus minimizing the likelihood of increasing the antigenicity of the molecule or inhibiting its ability to bind tissue factor; however two or more amino acid changes (substitutions, additions, or deletions) may be made and combinations of substitution(s), addition(s) and deletion(s) may also be made. In a preferred embodiment for human and bovine Factor VII, Ser344 is preferably substituted with Ala, but Gly, Met, Thr, or other amino acids can be substituted. It is preferred to replace Asp with Glu and to replace His with Lys or Arg. In general, substitutions are chosen to disrupt the tertiary protein structure as little as possible. One may introduce residue alterations as described above in the catalytic site of appropriate Factor VII sequence of human, bovine or other species and test the resulting protein for a desired level of inhibition of catalytic activity and resulting anticoagulant activity as described herein.

In preferred embodiments of human and bovine Factor VII, the active site residue Ser344 is modified, replaced with Gly, Met, Thr, or more preferably, Ala. Such substitution could be made separately or in combination with substitution(s) at other sites in the catalytic triad, which includes His193 and Asp242.

Examples of TF antagonists that can be used in accordance with the present invention include, but are not limited to:

Humanized TF-MAb blocking Factor X binding to the TF/FVIIa complex, CNTO-859 from Centocor (subsidiary of J&J); CNTO-859 is a humanized version of the murine TF-MAb, 5G8, developed by Scripps investigated for treatment of sepsis.

Murine TF-MAb, 5G8 from Scripps.

Humanized IgG1 kappa MAb that binds to TF, hOAT from Sunol, currently in preclinical studies for cancer.

Chimeric TF-MAb blocking FX binding to TF:FVIIa, cH36 from Sunol, currently in phase I for ALI, ARDS sepsis and coronary artery disease.

TF-MAb blocking FVIIa binding to TF, I43 from Sunol.

Humanized IgG4kappa MAb that binds to TF, hFAT from Sunol, investigated for the treatment of acute cardiovascular indications.

Humanized mouse monoclonal antibody to TF, preventing FX and FIX binding, hATR-5 from Chugai Pharmaceuticals.

Humanized anti-TF antibody F(ab′)2 fragment inhibiting FIX and FX binding to human TF:FVIIa, D3H44 from Genentech (Emory Univ.).

Recombinant humanized monoclonal antibody F(ab′)2 fragment that inhibits the TF/FVIIa complex from activating FIX and FX, PRO387474 from Genentech.

Dimer FFR-FVIIa and PEGylated FFR-FVIIa as disclosed in WO 02/02764 (University of Minnesota).

Immunoconjugate proteins constructed as a dimer of two identical chains, each having an effector domain which is the Fc region of an IgG1 immunoglobulin conjugated to a targeting domain which is a mutant form of factor VII that binds to tissue factor but does not initiate blood coagulation, as disclosed in WO 01/02439 (Garen).

Blood Qlucose Regulators:

In practicing the present invention, any compound may be used, which is able to regulate the blood glucose level. Blood glucose levels may be controlled by insulin treatment. However, it will be clear for the man skilled in the art that also active insulin derivatives and its physiologically tolerated salts and other blood glucose regulators can be used to obtain the same result. Furthermore, it will be clear for the man skilled in the art, that compounds of the group of biologically active substances having insulin releasing action can be used in practicing the present invention. Such compounds, capable of promoting the secretion of insulin, are known, e.g., the Islets-Activating Proteins (Ui; Michio et al. U.S. Pat. No. 5,000,953, 19 Mar. 1991) and the glucagon-like peptides (Habener; Joel F. Newton Highlands, MA, U.S. Pat. No. 5,614,492, 25 Mar. 1997).

Furthermore, it will be clear for the man skilled in the art, that compounds of the group of compounds that stimulate signal transduction mediated by an insulin receptor type tyrosine kinase in a cell can be used when practicing the present invention. It is known that insulin binding to the insulin receptor triggering a variety of metabolic and growth promoting effects. Metabolic effects include glucose transport, biosynthesis of glycogen and fats, inhibition of triglyceride breakdown, and growth promoting effects include DNA synthesis, cell division, and differentiation. It is known that some of these biological effects of insulin can be mimicked by vanadium salts such as vanadates and pervanadates. However, this class of compounds appears to inhibit phosphotyrosine phosphatases generally, and is potentially toxic because they contain heavy metal (U.S. Pat. No. 5,155,031; Fantus et al., 1989, Biochem. 28:8864-71; Swarup et al., 1982, Biochem. Biophys. Res. Commun. 107:1104-9). Furthermore, it has been demonstrated that certain protein-tyrosine phosphatases (PTPs or PTP's), in particular, RPTP.alpha and RPTP.epsilon., specifically regulate the insulin receptor signaling pathway (Lammers Reiner et al., 19 Jan. 1999, U.S. Pat. No. 5,861,266 and WO 9523217). ompounds that specifically modulate the activity of the controlling RPTP, thereby prolonging or enhancing signal transduction mediated by the insulin receptor can thus be used in accordance with the present invention. Such compounds have low toxicity since they are specific for the PTPs associated with insulin receptor activity, and do not significantly affect the activity of other PTPs that are non-specific.

Non-limiting examples of blood glucose regulators are insulin, active insulin derivatives, and insulin analogues, compounds that stimulate signal transduction mediated by an insulin receptor type tyrosine kinase in a cell, certain protein-tyrosine phosphatases (PTP's), other type II antidiabetica, and other biologically active substances having insulin releasing action.

The term “insulin”, as used herein, refers to insulin from any species such as porcine insulin, bovine insulin, and human insulin and salts thereof such as zinc salts, and protamine salts.

The term “active derivatives of insulin”, as used herein, are what a skilled art worker generally considers derivatives, including, for example, insulin having a substituent not present in the parent insulin molecule.

The term “insulin analogues”, as used herein refers to insulin wherein one or more of the amino acid residues have been exchanged with another amino acid residue and/or from which one or more amino acid residue has been deleted and/or from which one or more amino acid residue has been added with the proviso that said insulin analogue has a sufficient insulin activity. Using results from the so-called free fat cell assay, any skilled art worker, for example, a physician, knows when and which dosages to administer of the insulin analogue. Examples of insulin analogues are described in the following patents and equivalents thereto: U.S. Pat. No. 5,618,913, EP 254,516, EP 280,534, U.S. Pat. No. 5,750,497, and U.S. Pat. No. 6,011,007. Examples of specific insulin analogues are insulin aspart (i.e., AspB28 human insulin), insulin lispro (i.e., LysB28, ProB29 human insulin), and insulin glagin (i.e., GlyA21, ArgB31, ArgB32 human insulin).

Also compounds which can be considered being both an insulin derivative and an insulin analogue can be used to practice the present invention. Examples of such compounds are described in the following patents and equivalents thereto: U.S. Pat. No. 5,750,497, and U.S. Pat. No. 6,011,007. An example of a specific insulin analogues and derivatives is insulin detemir (i.e., des-ThrB30 human insulin gamma-LysB29 tetradecanoyl).

In the present context the three-letter or one-letter indications of the amino acids have been used in their conventional meaning as indicated in table 1. Unless indicated explicitly, the amino acids mentioned herein are L-amino acids. It is to be understood, that the first letter in, for example, K337 represent the amino acid naturally present at the indicated position wild-type factor VII, and that, for example, [K337A]-FVIIa designates the FVII-variant wherein the amino acid represented by the one-letter code K naturally present in the indicated position is replaced 10 by the amino acid represented by the one-letter code A.

TABLE 1
Abbreviations for amino acids:

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |

The term “factor VIIa” or “FVIIa” may be used interchangeably.

The term “systemic inflammatory response syndrome (SIRS)”, as used herein refers to the uncontrolled disease process which ensues upon an initial insult and which gives rise to a multisystem disturbance secondary to inflammatory mediators released during shock.

The term “sepsis”, as used herein refers to “SIRS”, as described above, which is particularly caused by an infectious insult leading to the initial shock phase.

The term “mediators of sepsis”, as used herein refers to factors released by inflammatory cells, such as TNFs, interleukins, bradykinins etc.

The term “insulin receptor type tyrosine kinase”, as used herein refers to a post-receptor signal transduction pathway involved in the insulin signaling.

The term “endoneural edema”, as used herein refers to swelling of the neuronal cells.

The term “phrenic nerves”, as used herein refers to the left and right nervus phrenicus,

The term “non-diabetic patient”, as used herein refers to a patient who has not been diagnosed as having diabetes.

In its broadest sense, the term a “critically ill patient” (herein designated CIP), as used herein refers to a patient who has sustained or are at risk of sustaining acutely life-threatening single or multiple organ system failure due to disease or injury, a patient who is being operated and where complications supervene, and a patient who has been operated in a vital organ within the last week or has been subject to major surgery within the last week. In a more restricted sense, the term a “critically ill patient”, as used herein refers to a patient who has sustained or are at risk of sustaining acutely life-threatening single or multiple organ system failure due to disease or injury, or a patient who is being operated and where complications supervene. In an even more restricted sense, the term “critically ill patient”, as used herein, refers to a patient who has sustained or are at risk of sustaining acutely life-threatening single or multiple organ system failure due to disease or injury. Similarly, these definitions apply to similar expressions such as “critical illness in a patient” and a “patient is critically ill”.

The term “Intensive Care Unit” (herein designated ICU), as used herein refers to the part of a hospital where critically ill patients are treated. Of course, this might vary from country to country and even from hospital to hospital and said part of the hospital may not necessary, officially, bear the name “Intensive Care Unit” or a translation or derivation thereof. Of course, the term “Intensive Care Unit” also covers a nursing home, a clinic, for example, a private clinic, or the like if the same or similar activities are performed there.

The term “active site” and the like when used herein with reference to FVIIa, refers to the catalytic and zymogen substrate binding site, including the “S1” site of FVIIa as that term is defined by Schecter, I. and Berger, A, (1967) Biochem. Biophys. Res. Commun. 7:157-162.

The term “TF-mediated coagulation activity” means coagulation initiated by TF through the formation of the TF/FVIIa complex and its activation of FIX and Factor X to FIXa and FXa, respectively. TF-mediated coagulation activity is measured in an FXa generation assay. The term “FXa generation assay” as used herein is intended to mean any assay where activation of FX is measured in a sample comprising TF, FVIIa, FX, calcium and phospholipids. Examples of FXa generation assays are described in assay 1 and assay 2 (of the present specification).

A TF/FVIIa mediated or associated process or event, or a process or event associated with TF-mediated coagulation activity, is any event, which requires the presence of TF/FVIIa.

Such processes or events include, but are not limited to, formation of fibrin which leads to thrombus formation; platelet deposition; proliferation of smooth muscle cells (SMCs) in the vessel wall, such as, for example, in intimal hyperplasia or restenosis, which is thought to result from a complex interaction of biological processes including platelet deposition and thrombus formation, release of chemotactic and mitogenic factors, and the migration and proliferation of vascular smooth muscle cells into the intima of an arterial segment; and deleterious events associated with post-ischemic reperfusion, such as, for example, in patients with acute myocardial infarction undergoing coronary thrombolysis.

The general mechanism of blood clot formation is reviewed by Ganong in “Review of Medical Physiology, 13th ed” (Lange, Los Altos Calif., pp 411-414 (1987)). Coagulation requires the confluence of two processes, the production of thrombin which induces platelet aggregation and the formation of fibrin which renders the platelet plug stable. The process comprises several stages each requiring the presence of discrete proenzymes and profactors. The process ends in fibrin crosslinking and thrombus formation. Fibrinogen is converted to fibrin by the action of thrombin. Thrombin, in turn, is formed by the proteolytic cleavage of prothrombin. This proteolysis is effected by factor Xa which binds to the surface of activated platelets and in the presence of FVa and calcium, cleaves prothrombin. TF/FVIIa is required for the proteolytic activation of factor X by the extrinsic pathway of coagulation. Therefore, a process mediated by or associated with TF/FVIIa, or a TF-mediated coagulation activity includes any step in the coagulation cascade from the formation of the TF/FVIIa complex to the formation of a fibrin platelet clot and which initially requires the presence of TF/FVIIa. For example, the TF/FVIIa complex initiates the extrinsic pathway by activation of factor X to factor Xa, FIX to FIXa, and additional FVII to FVIIa.

A TF/FVIIa mediated or associated process, or TF-mediated coagulation activity, can be conveniently measured employing standard assays such as, e.g., those described in Roy, S., (1991) J. Biol. Chem. 266:4665-4668, and O'Brien, D. et al., (1988) J. Clin. Invest. 82:206-212 for the conversion of factor X to factor Xa in the presence of TF/FVIIa and other necessary reagents.

The term “TF related diseases or disorders” as used herein means any disease or disorder, where TF is involved. This includes, but are not limited to diseases or disorders related to TF-mediated coagulation activity, thrombotic or coagulopathic related diseases or disorders or diseases or disorders such as inflammatory responses and chronic thromboembolic diseases or disorders associated with fibrin formation, including vascular disorders such as deep venous thrombosis, arterial thrombosis, post surgical thrombosis, coronary artery bypass graft (CABG), percutaneous transdermal coronary angioplastry (PTCA), stroke, tumor metastasis, angiogenesis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, and other diseases. The TF related diseases or disorders are not limited to in vivo coagulopatic disorders such as those named above, but includes ex vivo TF/FVIIa related processes such as coagulation that may result from the extracorporeal circulation of blood, including blood removed in-line from a patient in such processes as dialysis procedures, blood filtration, or blood bypass during surgery.

It should be noted that peptides, proteins and amino acids as used herein can comprise or refer to “natural”, i.e., naturally occurring amino acids as well as “non-classical” D-amino acids including, but not limited to, the D-isomers of the common amino acids, alpha-isobutyric acid, 4-aminobutyric acid, hydroxyproline, sarcosine, citrulline, cysteic acid, t-butylglycine, t-butylalanine, phenylglycine, cyclohexylalanine, beta-alanine, designer amino acids such as beta-methyl amino acids, C-alpha-methyl amino acids, N-alpha-methyl amino acids, and amino acid analogues in general. In addition, the amino acids can include Abu, 2-amino butyric acid; gamma-Abu, 4-aminobutyric acid; epsilon-Ahx, 6-aminohexanoic acid; Aib, 2-amino-isobutyric acid; beta-Ala, 3-aminopropionic acid; Orn, ornithine; Hyp, trans-hydroxyproline; Nle, norleucine; Nva, norvaline.

The three-letter indication “GLA’ as used herein means 4-carboxyglutamic acid (gamma-carboxyglutamate).

The terms “human tissue factor” or “human TF” as used herein, refers to the full length polypeptide receptor comprising the amino acid sequence 1-263 of native human tissue factor.

The term ‘antibody’, as used herein, is intended to refer to immunoglobulin molecules and fragments thereof, which have the ability to specifically bind to an antigen (e.g., human TF). Full-length antibodies comprises four polypeptide chains, two heavy (H) chains and two light (L) chains interconnected by disulfide bonds, Each heavy chain is comprised of a heavy chain variable region (abbreviated herein as HCVR or VH) and a heavy chain constant region. The heavy chain constant region is comprised of three domains, CH1, CH2, and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDRI, FR2, CDR2, FR3, CDR3, FR4.

Thus, within the definition of an antibody are also one or more fragments of an antibody that retain the ability to specifically bind to an antigen (e.g., human TF). It has been shown that the antigen-binding function of an antibody can be performed by fragments of a full-length antibody. Examples of binding fragments encompassed within the term “antibody” include (i) a Fab fragment, a monovalent fragment consisting of the VL, VH, CL and CH1 domains; (ii) F(ab)2 and F(ab′)2 fragments, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426: and Huston et al. (1988) Proc. Natl. Acad. Sci. USA 85:5879-5883). Such single chain antibodies are also intended to be encompassed within the term “antibody”. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak. R. J., et al. (1994) Structure 2:1121-1123). It is understood that human TF may have one or more antigenic determinants comprising (1) peptide antigenic determinants which consist of single peptide chains within human TF, (2) conformational antigenic determinants which consist of more than one spatially contiguous peptide chains whose respective amino acid sequences are located disjointedly along the human TF polypeptide sequence; and (3) post-translational antigenic determinants which consist, either in whole or part, of molecular structures covalently attached to human TF after translation, such as carbohydrate groups, or the like.

The terms “human antibody”, “human antibodies”, “human TF antibody”, and “human TF antibodies”, as used herein, is intended to include antibodies having variable and constant regions derived from human germ line immunoglobulin sequences. The human antibodies of the invention may include amino acid residues not encoded by human germ line immunoglobulin sequences (e.g., mutations introduced by random or site-specific mutagenesis in vitro or by somatic mutation in vivo), for example in the CDRs and in particular CDR3. The term “humanized antibody”, as used herein, is intended to include antibodies in which CDR sequences derived from the germ line of another mammalian species, such as a mouse, have been grafted onto human framework sequences; the term “humanized antibody” can be used interchangeably with “chimeric antibody”, and “chimera antibodies”, (e.g., human/mouse chimera antibodies”).

By “catalytically inactivated in the active site of the FVIIa polypeptide” is meant that a factor VIIa inhibitor is bound to the factor VIIa polypeptide and decreases or prevents the factor VIIa-catalyzed conversion of factor X to factor Xa. An FVIIa inhibitor may be identified as a substance, which reduces the amidolytic activity by at least 50% at a concentration of the substance at 400 micromolar (UM) in the FVIIa amidolytic assay described by Persson et al. (Persson et al., J. Biol. Chem. 272: 19919-19924 (1997)). Preferred are substances reducing the amidolytic activity by at least 50% at a concentration of the substance at 300 μM; more preferred are substances reducing the amidolytic activity by at least 50% at a concentration of the substance at 200 μM.

The “FVIIa inhibitor” may be selected from any one of several groups of FVIIa directed inhibitors. Such inhibitors are broadly categorized for the purpose of the present invention into i) inhibitors which reversibly bind to FVIIa and are cleavable by FVIIa, ii) inhibitors which reversibly bind to FVIIa but cannot be cleaved, and iii) inhibitors which irreversibly bind to FVIIa. For a review of inhibitors of serine proteases see Proteinase Inhibitors (Research Monographs in cell and Tissue Physiology; v. 12) Elsevier Science Publishing Co., Inc., New York (1990).

The FVIIa inhibitor moiety may also be an irreversible FVIIa serine protease inhibitor. Such irreversible active site inhibitors generally form covalent bonds with the protease active site. Such irreversible inhibitors include, but are not limited to, general serine protease inhibitors such as peptide chloromethylketones (see, Williams et al., J. Biol. Chem. 264:7536-7540 (1989)) or peptidyl cloromethanes; azapeptides; acylating agents such as various guanidinobenzoate derivatives and the 3-alkoxy-4-chloroisocoumarins; sulphonyl fluorides such as phenylmethylsulfonyl-fluoride (PMSF); diisopropylfluorophosphate (DFP); tosylpropylchloromethyl ketone (TPCK); tosyllysylchloromethyl ketone (TLCK); nitrophenylsulphonates and related compounds; heterocyclic protease inhibitors such as isocoumarines, and coumarins.

Examples of peptidic irreversible FVIIa inhibitors include, but are not limited to, Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone.

Examples of FVIIa inhibitors also include benzoxazinones or heterocyclic analogues thereof such as described in PCT/DK99/00138.

Examples of other FVIIa inhibitors include, but are not limited to, small peptides such as for example Phe-Phe-Arg, D-Phe-Phe-Arg, Phe-Phe-Arg, D-Phe-Phe-Arg, Phe-Pro-Arg, D-Phe-Pro-Arg. Phe-Pro-Arg, D-Phe-Pro-Arg, L- and D-Glu-Gly-Arg; peptidomimetics; benzamidine systems; heterocyclic structures substituted with one or more amidino groups; aromatic or heteroaromatic systems substituted with one or more C(═NH)NHR groups in which R is H, C1-3alkyl. OH or a group which is easily split of in vivo.

Thrombotic or coagulopathic related diseases or disorders: The term includes vascular diseases and inflammatory responses including, without limitation, deep venous thrombosis, arterial thrombosis, post surgical thrombosis, coronary artery bypass graft (CABG), percutaneous transdermal coronary angioplastry (PTCA), stroke, tumor metastasis, inflammation, septic chock, hypotension, acute lung injury (ALI), Acute Respiratory Distress Syndrome (ARDS), pulmonary embolism, disseminated intravascular coagulation (DIC), sepsis, systemic inflammatory response syndrome (SIRS), vascular restenosis, platelet deposition, myocardial infarction, angiogenesis, or the treatment of mammals with atherosclerotic vessels at risk for thrombosis, and multiple organ failure resulting from any of the preceding pathologic processes.

Respiratory Diseases or disorders: Exemplified by lower respiratory diseases such as systemic inflammatory response syndrome, asthma, bronchitis, acute lung injury, acute respiratory distress syndrome, idiopathic pulmonary fibrosis, pneumonia, pulmonary edema, pulmonary obstructive disease, endotoxin induced lung damage, non cell lung cancer, and multiple organ failure resulting from any of the preceding pathologic processes.

Inflammatory Diseases or disorders: Refers to diseases such as inflammatory bowel disease, sepsis, septic shock, acute respiratory distress syndrome, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondyarthropathris, ankylosing spondylitis, Reiter's syndrome, psoriatic arthropathy, enterapathric spondylitis, juvenile arthropathy or juvenile ankylosing spondylitis, reactive arthropathy, infectious or post-infectious arthritis, gonoccocal arthritis, tuberculous arthritis, viral arthritis, fungal arthritis, syphilitic arthritis, Lyme disease, arthritis associated with vasculitic syndromes, polyarteritis nodosa, hypersensitivity vasculitis, Luegenec's granulomatosis, polymyalgin rheumatica, joint cell arteritis, calcium crystal deposition arthropathris, pseudo gout, non-articular rheumatism, bursitis, tenosynomitis, epicondylitis (tennis elbow), carpal tunnel syndrome, repetitive use injury (e.g., from typing), miscellaneous forms of arthritis, neuropathic joint disease (charco and joint), hemarthrosis (hemarthrosic), Henoch-Schonlein Purpura, hypertrophic osteoarthropathy, multicentric reticulohistiocytosis, arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, relapsing, and multiple organ failure resulting from any of the preceding pathologic processes.

The phrase “therapeutically effective interval” is a period of time beginning when one of either (a) the TF antagonist or (b) The blood glucose regulator is administered to a mammal and ending at the limit of the beneficial effect in preventing or ameliorating respiratory or inflammatory disease or associated CIP and/or organ failure of (a) or (b).

“Sole” agents or factors, as used herein, refers to situations in which the TF antagonist and the blood glucose regulator, taken together, are the only haemostatic agents, or active haemostatic agents, or coagulation factors contained in the pharmaceutical composition or kit, or are the only haemostatic agents, or active haemostatic agents, or coagulation factors administered to the patient in the course of a particular treatment, such as, e.g., in the course of a particular bleeding episode. It will be understood that these situations encompass those in which other haemostatic agents or coagulation factors, as applicable, are not present in either sufficient quantity or activity so as to significantly influence one or more coagulation parameter.

Clot lysis time, clot strength, fibrin clot formation, and clotting time are clinical parameters used for assaying the status of patient's haemostatic system. Blood samples are drawn from the patient at suitable intervals and one or more of the parameters are assayed by means of, e.g., thromboelastograpy as described by, e.g., Meh et al., Blood Coagulation & Fibrinolysis 2001; 12:627-637; Vig et al.. Hematology, Vol. 6 (3) pp. 205-213 (2001); Vig et al., Blood coagulation & Fibrinolysis, Vol.12 (7) pp. 555-561 (2001) Oct; Glidden et al., Clinical and applied thrombosis/hemostasis, Vol. 6 (4) pp. 226-233 (2000) Oct; McKenzie et al., Cardiology, Vol. 92 (4) pp. 240-247 (1999) Apr; or Davis et al., Journal of the American Society of Nephrology, Vol. 6 (4) pp. 1250-1255 (1995).

In this context, the term “treatment” is meant to include both prevention of an expected unwanted clotting, and regulation of an already occurring clotting. Prophylactic administration of a preparation of a TF antagonist and a blood glucose regulator is thus included in the term “treatment”.

The term “subject” as used herein is intended to mean any animal, in particular mammals, such as humans, and may, where appropriate, be used interchangeably with the term “patient”.

The TF antagonist and the blood glucose regulator as defined in the present specification may be administered simultaneously or sequentially. The factors may be supplied in single-dosage form wherein the single-dosage form contains both coagulation factors, or in the form of a kit-of-parts comprising a preparation of a TF antagonist as a first unit dosage form and a preparation of a blood glucose regulator as a second unit dosage form. Whenever a first or second or third, etc., unit dose is mentioned throughout this specification this does not indicate the preferred order of administration, but is merely done for convenience purposes.

By “simultaneous” dosing of a preparation of a TF antagonist and a preparation of a blood glucose regulator is meant administration of the coagulation factor proteins in single-dosage form, or administration of a first coagulation factor protein followed by administration of a second coagulation factor protein with a time separation of no more than 15 minutes, preferably 10, more preferred 5, more preferred 2 minutes. Either factor may be administered first.

By “sequential” dosing is meant administration of a first coagulation factor protein followed by administration of a second coagulation factor protein with a time separation of more than 15 minutes. Either of the two unit dosage form, or coagulation factor proteins, may be administered first. Preferably, both products are injected through the same intravenous access.

By “APTT” or “aPTT” is meant the activated partial thromboplastin time (described by, e.g., Proctor RR, Rapaport SI: The partial thromboplastin time with kaolin; a simple screening test for first-stage plasma clotting factor deficiencies. Am J Clin Pathol 36:212, 1961).

“Half-life” refers to the time required for the plasma concentration of a TF antagonist or a blood glucose regulator to decrease from a particular value to half of that value.

The total amount of protein in a preparation may be measured by generally known methods, e.g., by measuring optical density. Amounts of proteins (“antigen”) may be measured by generally known methods such as standard Elisa immuno assays. In general terms, such assay is conducted by contacting. e.g., a solution of the factor VII-containing preparation with an anti-FVII antibody immobilized onto the Elisa plate, subsequently contacting the immobilized antibody-FVII complex with a second anti-FVII antibody carrying a marker, the amounts of which, in a third step, are measured. The amounts of each polypeptide present may be measured in a similar way using appropriate antibodies. The total amount of protein present in a preparation is determined by adding the amounts of the individual proteins. In one embodiment, the preparation comprises isolated coagulation factors. In another embodiment the preparation is free of coagulation factor II and coagulation factor IIa (prothrombin and thrombin) and/or factor X or Xa.

As used herein, the term “isolated” refers to polypeptides, e.g., insulin, that have been separated from the cell in which they were synthesized or the medium in which they are found in nature (e.g., plasma or blood). Separation of polypeptides from their cell of origin may be achieved by any method known in the art, including, without limitation, removal of cell culture medium containing the desired product from an adherent cell culture; centrifugation or filtration to remove non-adherent cells; and the like. Separation of polypeptides from the medium in which they naturally occur may be achieved by any method known in the art, including, without limitation, affinity chromatography, such as, e.g., on an anti-factor VII antibody column; hydrophobic interaction chromatography; ion-exchange chromatography; size exclusion chromatography; electrophoretic procedures (e.g., preparative isoelectric focusing (IEF)), differential solubility (e.g., ammonium sulfate precipitation), or extraction and the like.

Within the present invention an “effective amount” of a TF antagonist and a blood glucose regulator is defined as the amount of a TF antagonist, e.g., inactivated FVIIa or a TF antibody, and a blood glucose regulator that together suffices to cure, alleviate or partially arrest the disease and its complications.

The phrase “therapeutically effective combination”, used in the practice of this invention, means administration of both (a) a TF antagonist and (b) a blood glucose regulator, either simultaneously or separately.

The term, “Active Ingredient” as used herein refers to a combination of (a) a TF antagonist and (b) a blood glucose regulator co-present in a pharmaceutical formulation for the delivery of a treatment regimen that applies this invention.

The term, “injectable liquid carrier” refers to a liquid medium containing either or both of (a) a TF antagonist, or (b) a blood glucose regulator; wherein (a) and (b) are independently dissolved, suspended, dispersed, or emulsified in the liquid medium.

Abbreviations

TF tissue factor

FVII factor VII in its single-chain, unactivated form

FVIIa factor VII in its activated form

rFVIIa recombinant factor VII in its activated form

APTT activated partial thromboplastin time.

Preparation of Compounds:

Methods for preparing recombinant proteins including conventional molecular biology, microbiology, and recombinant DNA techniques are within the skill of the art. Such techniques are explained fully in the literature. See. e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”) DNA Cloning: A Practical Approach, Volumes I and II (D.N. Glover ed. 1985); Oligonucleotide Synthesis (M. J. Gait ed. 1984); Nucleic Acid Hybridization (B. D. Hames & S. J. Higgins, eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

Briefly, DNA sequences encoding a specific protein (e.g., insulin or human FVII) may be isolated by preparing a genomic or cDNA library and screening for DNA sequences coding for all or part of the protein by hybridization using synthetic oligonucleotide probes in accordance with standard techniques (cf. Sambrook et al., above). For the present purpose, the DNA sequence encoding the protein is preferably of human origin, i.e. derived from a human genomic DNA or cDNA library.

Polypeptide variants may be made by amino acid sequence alterations of the polypeptide, which may be accomplished by a variety of techniques. Modification of the DNA sequence may be by site-specific mutagenesis. Techniques for site-specific mutagenesis are well known in the art and are described by, for example, Zoller and Smith (DNA 3:479-488, 1984).

The DNA sequences encoding the polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

The DNA sequences may also be prepared by polymerase chain reaction using specific primers, for instance as described in U.S. Pat. No. 4,683,202, Saiki et al., Science 239 (1988), 487-491, or Sambrook et al., supra.

The host cell into which the DNA sequences encoding the polypeptides is introduced may be any cell, which is capable of producing the posttranslational modified human FVII polypeptides and includes yeast, fungi and higher eukaryotic cells. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725;

Corsaro and Pearson, Somatic Cell Genetics 7 (1981), 603; Graham and van der Eb, Virology 52 (1973), 456; and Neumann et al., EMBO J. 1 (1982), 841-845.

Methods for producing antibodies and fragment of antibodies are generally known in the art, see, e.g., Harboe and Ingild, In N. H. Axelsen, J. Kroll, and B. Weeks, editors, A Manual of Quantitative Immunoelectrophoresis, Blackwell Scientific Publications, 1973, Chapter 23, or Johnstone and Thorpe, Immunochemistry in Practice, Blackwell Scientific Publications, 1982 (more specifically pages 27-31). Preferably, the antibodies are monoclonal antibodies. Monoclonal antibodies may be prepared, e.g., according to the methods of E. Harlow and D. Lane, editors, 1988, Antibodies, A Laboratory Manual, Cold Spring Harbor Press, Cold Spring Harbor, N.Y.

Methods of preparing human antibodies against human TF are, for example, described in International application No. PCT/DK02/00644.

Factor VII Polypeptides:

For the preparation of recombinant human FVII polypeptides, a cloned wild-type FVII DNA sequence is used. This sequence may be modified to encode a desired FVII variant. The complete nucleotide and amino acid sequences for human FVII are known; see U.S. Pat. No. 4,784,950, wherein the cloning and expression of recombinant human FVII is described. The bovine FVII sequence is described in Takeya et al., J. Biol. Chem, 263:14868-14872 (1988), which is incorporated by reference herein.

DNA sequences for use will typically encode a pre-pro peptide at the amino-terminus of the FVII protein to obtain proper post-translational processing (e.g. gamma-carboxylation of glutamic acid residues) and secretion from the host cell. The pre-pro peptide may be that of FVII or another vitamin K-dependent plasma protein, such as factor IX, factor X, prothrombin, protein C or protein 5. As will be appreciated by those skilled in the art, additional modifications can be made in the amino acid sequence of FVII where those modifications do not significantly have impact on the ability of the protein to act as a coagulation factor. For example, FVII modified in the catalytic triad can also be modified in the activation cleavage site to inhibit the conversion of zymogen FVII into its activated two-chain form, as generally described in U.S. Pat. No. 5,288,629.

Factor VII polypeptides for use in the present invention may be prepared, e.g., as described in International Applications Nos. WO 92/15686. WO 94/27631 and WO 96/12800; Wildgoose et al., Biochem 29:3413-3420, 1990; Kazama et al., J. Biol. Chem. 27056-72, 1995; Holst et al., Eur. J. Vasc. Endovasc. Surg. 15:515-520, 1998; and Nicolaisen et al., FEBS Letts. 317:245-249, 1993.

FVII polypeptides produced as described above may be purified by affinity chromatography on an anti-FVII antibody column. The immunoadsorption column comprises a high-specificity monoclonal antibody, such as, e.g., a calcium-dependent monoclonal antibody as described by Wakabayashi et al., J. Biol. Chem, 261:11097-11108, (1986) and Thim et al., Biochem. 27: 7785-7793, (1988). Additional purification may be achieved by conventional chemical purification means, such as high performance liquid chromatography. Other methods of purification, including barium citrate precipitation, are known in the art, and may be applied to the purification of the FVII described herein (see, generally, Scopes, R., Protein Purification, Springer-Verlag, N.Y., 1982).

Conversion of single-chain FVII to active two-chain FVIIa may be achieved using factor XIIa as described by Hedner and Kisiel (1983, J. Clin. Invest. 71: 1836-1841), or using other proteases having trypsin-like specificity (Kisiel and Fujikawa, Behring Inst. Mitt. 73: 29-42, 1983).

Alternatively FVII may be autoactivated by passing it through an ion-exchange chromatography column such as mono Q.RTM. (Pharmacia Fire Chemicals) or the like (Bjoern et al., 1986, Research Disclosures 269564-565).

Preparation of the Glucose Regulating Agent

The preparation of insulin, e.g., pig insulin and human insulin, whether isolated from glands or recombinantly produced, is well known to the skilled worker. Reference to publications describing different types of insulin analogues and derivatives, as well as alternative types of glucose regulating agents are given supra.

Pharmaceutical Compositions and Methods of Use

The preparations of the present invention may be used to treat thrombotic and coagulopathic related diseases or disorders, respiratory diseases or disorders, and inflammatory diseases or disorders including, without limitation, deep venous thrombosis, arterial thrombosis, post surgical thrombosis, coronary artery bypass graft (CABG), percutaneous transdermal coronary angioplastry (PTCA), stroke, tumor metastasis, inflammation, septic chock, hypotension, acute lung injury (ALI), Acute Respiratory Distress Syndrome (ARDS), pulmonary embolism, disseminated intravascular coagulation (DIC), sepsis, systemic inflammatory response syndrome (SIRS), vascular restenosis, platelet deposition, myocardial infarction, angiogenesis, or the prophylactic treatment of mammals with atherosclerotic vessels at risk for thrombosis; asthma, bronchitis, idiopathic pulmonary fibrosis, pneumonia, pulmonary edema, pulmonary obstructive disease, endotoxin induced lung damage, non cell lung cancer; inflammatory bowel disease, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondylarthropathris, ankylosing spondylitis, Reiter's syndrome, psoriatic arthropathy, enterapathric spondylitis, juvenile arthropathy or juvenile ankylosing spondylitis, reactive arthropathy, infectious or post-infectious arthritis, gonoccocal arthritis, tuberculous arthritis, viral arthritis, fungal arthritis, syphilitic arthritis, Lyme disease, arthritis associated with “vasculitic syndromes” polyarteritis nodosa, hypersensitivity vasculitis, Luegenec's granulomatosis, polymyalgin rheumatica, joint cell arteritis, calcium crystal deposition arthropathris, pseudogout, non-articular rheumatism, bursitis, tenosynomitis, epicondylitis (tennis elbow), carpal tunnel syndrome, repetitive use injury (typing). miscellaneous forms of arthritis, neuropathic joint disease (charco and joint), hemarthrosis (hemarthroic), Henoch-Schonlein Purpura, hypertrophic osteoarthropathy, multicentric reticulohistiocytosis, arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, relapsing, and multiple organ failure resulting from any of the preceding pathologic processes.

The essential ingredients (a) a TF antagonist and (b) a blood glucose regulator are present in the formulation in such proportion that a dose of the formulation provides an amount of each ingredient that together is a pharmaceutically effective amount to the patient being treated. The dose of composition of the invention to be administered is determined depending upon age, body weight, symptom, the desired therapeutic effect, the route of administration, and the duration of the treatment etc. Typically, the weight ratio of TF antagonist and the amount of blood glucose regulator (e.g., insulin or a biologically active fragment or variant thereof) may vary from a ratio of between about 1:100 to about 100:1 (w/w). The ratio of TF antagonist to blood glucose regulator (e.g., insulin or biologically active fragment or variant) may thus be, e.g., about 1:100, or 1:90, or 1:80 or 1:70 or 1:60, or 1:50 or 1:40, or 1:30, or 1:20, or 1:10, or 1:5, or 1:2, or 1:1, or 2:1, or 5:1, or 10:1, or 20:1, or 30:1, or 40:1, or 50:1, or 60:1, or 70:1, or 80:1, or 90:1, or 100:1; or between about 1:90 to about 1:1, or between about 1:80 to about 1:2, or between about 1:70 to about 1:5, or between about 1:60 to about 1:10, or between about 1:50 to about 1:25, or between about 1:40 to about 1:30, or between about 90:1 to about 1:1, or between about 80:1 to about 2:1, or between about 70:1 to about 5:1, or between about 60:1 to about 10:1, or between about 50:1 to about 25:1, or between about 40:1 to about 30:1; or between about 10:1 to about 1:10, or between about 5:1 to about 1:5.

Whenever a first or second or third, etc., unit dose is mentioned throughout this specification this does not indicate the preferred order of administration, but is merely done for convenience purposes.

The dose of the TF antagonist ranges from about 0.05 to about 500 mg/day; e.g., from about 1 to about 200 mg/day, or, e.g., from about 5 to about 175 mg/day for a 70-kg subject as loading and maintenance doses, depending on the weight of the subject, the condition and the severity of the condition.

The dose of the blood glucose regulator is regulated in such a way that the blood glucose level is reduced. Preferably, the blood glucose regulator, e.g., insulin, is capable of maintaining blood glucose at or below 130 mg per deciliter patient plasma, preferably at or below 110 mg/dl. It is however preferred that the blood glucose levels are regulated and maintained within a range where the lower limit can be selected to be about 60, about 70 or about 80 mg/dL and the upper limit can be selected to be about 110, about 120 or about 130 mg/dL, more specifically to the normal range (i.e., from about 80 to about 110 mg/dL). The skilled art worker, for example, the physician, will be able to decide exactly which upper and lower limits to use. Alternatively, the range is from about 60 to about 130, preferably, from about 70 to about 120, more preferred, from about 80 to about 110 mg/dL.

It must be kept in mind that the materials of the present invention may generally be employed in serious disease or injury states, that is, life threatening or potentially life threatening situations. In such cases, in view of the minimization of extraneous substances and general lack of immunogenicity of factor VIIa and insulin in humans, it is possible and may be felt desirable by the treating physician to administer a substantial excess of these compositions. A dose may be given continuously or intermittently (once or several times a day). In making compositions of the invention the essential ingredients; TF antagonist and blood glucose regulator are co-present and may be mixed in any homogeneous or non-homogeneous manner or adjacently or otherwise proximately placed together in an individual dosage unit suitable for practicing the method of the invention. The dosage unit of the TF antagonist and/or blood glucose regulator will usually be admixed with a carrier or inert ingredients, or diluted by a carrier, or enclosed within a carrier which may be in the form of an ampoule (e.g., for use in a pen device, a pump device, or other injection or infusion device), capsule, time release dosing device, sachet, paper or other container. When the carrier serves as a diluent, it may be a solid, semi-solid, paste, or liquid material which acts as a vehicle, or can be in the form of tablets, pills, powders, lozenges, elixirs, suspensions, emulsions, solutions, syrups, aerosols (as a solid or in a liquid medium), or ointment, containing, for example, up to 10% by weight of the active compound. The dosage unit of the active ingredients will usually be admixed with a liquid carrier and/or other inert ingredients or enclosed within a carrier which may be in the form of an ampoule, bottle, time release dosing device or other container. When the carrier serves as a diluent, it may be a liquid material which acts as a vehicle, or can be in the form of solutions containing, for example, up to 10% by weight of the active compound. For the pharmaceutical formulations containing both (a) TF antagonist and (b) a blood glucose regulator the carrier may be an injectable liquid medium such as is well known in the art. The injectable liquid must be such that permits parenteral administration, that is, introduction of substances to a mammal being treated by intervenous, subcutaneous, intramuscular, or intramedullary injection. Intravenous injection is most preferred as a means of administration. The Active ingredient can be dissolved or suspended in a pharmaceutically acceptable carrier, such as sterile water, sterile water containing saline and/or sugars and/or suspension agents or a mixture of both. For example, for intravenous injection the compounds of the invention may be dissolved at a concentration of about 2 mg/ml in a 4% dextrose/0.5% sodium citrate aqueous solution. Liquid compositions for oral administration include pharmaceutically-acceptable emulsions, solutions, suspensions, syrups, and elixirs containing inert diluents commonly used in the art such as distilled water or ethanol. Besides inert diluents such compositions may also comprise adjuvants such as wetting and suspending agents, and sweetening, flavoring, perfuming and preserving agents. Other compositions for oral administration include spray compositions which may be prepared by known methods and which comprise one or more of the active compound(s). Besides inert diluents such compositions may also comprise stabilizers such as sodium bisulfite and buffer for isotonicity, for example sodium chloride, sodium citrate, or citric acid. The manufacturing methods of spray compositions for inhalation therapy are described in detail in the art. Preparations for injection according to the present invention for parenteral administration include sterile aqueous or non-aqueous solutions, suspensions, or emulsions. Examples of aqueous solvents or suspending media are distilled water for injection and physiological salt solution. Examples of non-aqueous solvents or suspending media are propylene glycol, polyethylene glycol, vegetable oils such as olive oil, alcohols such as ethanol, Polysorbate 80 (registered Trade Mark). These compositions may also include adjuvants such as preserving, wetting, emulsifying and dispersing agents stabilizing agents (e.g. lactose) and solubilizers (e.g. glutamic acid and asparaginic acid).They may be sterilized, for example, by filtration through a bacteria-retaining filter, by incorporation of sterilizing agents in the compositions or by irradiation. They may also be manufactured in the form of sterile solid compositions which can be dissolved in sterile water or some other sterile injectable medium immediately before use.

The TF antagonist and/or the blood glucose regulator may be in the form of powder, tablet, or capsule. A solid carrier can be one or more substances which may also act as flavoring agents, lubricants, solubilizers, suspending agents, binders, tablet disintegrating agents, and encapsulating material. Suitable solid carriers are magnesium carbonate, magnesium stearate, talc, sugar lactose, pectin, dextrin, starch, gelatin, tragacanth, methyl cellulose, sodium carboxymethyl cellulose, low melting waxes, and cocoa butter. Preferably, the pharmaceutical compositions are administered parenterally, i.e., intravenously, subcutaneously, or intramuscularly; intravenously being most preferred. They may also be administered by continuous or pulsatile infusion. Local delivery of the preparations of the present invention, such as, for example, topical application, may be carried out, e.g., by means of a spray, perfusion, double balloon catheters, stents, incorporated into vascular grafts or stents, hydrogels used to coat balloon catheters, or other well established methods. One skilled in this art may formulate the compositions of the invention an appropriate manner, and in accordance with accepted practices, such as those disclosed in Remington's Pharmaceutical Sciences, Gennaro, ed., Mack Publishing Co., Easton, Pa., 1990.

This invention is a method of treating or preventing thrombotic and coagulopathic diseases, Inflammatory Disease or Respiratory Disease by administering to a mammal in need thereof a therapeutically effective amount of (a) a TF antagonist and b) a blood glucose regulator; wherein (a) and (b) are both administered within a therapeutically effective interval. The administration of (a) or (b) to, e.g., a septic or CIP patient may be either continuous or intermittent.

The blood glucose regulator and the TF antagonist can be delivered simultaneously. One convenient method of simultaneous delivery is to use the compositions of the invention, wherein the Active Ingredient has the essential ingredients co-present in a unit dosage form. Solutions or suspensions of mixed essential ingredients may, if desired, be delivered from the same liquid holding bag. Another method of simultaneous delivery is to deliver the two compounds to the patient separately but simultaneously. Thus, for example, some TF antagonists may be given as an oral formulation at the same time as a blood glucose regulator is given parenterally. Dosage of a TF antagonist can begin simultaneously with the blood glucose regulator administration. The duration of the TF antagonist administration can extend past the blood glucose regulator administration, or vice versa. It is however preferred that the blood glucose regulator is administered first; the level of blood glucose is monitored, and when the level of blood glucose has been reduced to a level at or below 130 mg per deciliter of patient plasma, preferably at or below 110 mg/dl, the TF antagonist is administered to the patient. It is preferred that the blood glucose levels are regulated and maintained within a range where the lower limit can be selected to be about 60, about 70 or about 80 mg/dL and the upper limit can be selected to be about 110, about 120 or about 130 mg/dL, more specifically to the normal range (i.e., from about 80 to about 110 mg/dL). The skilled art worker, for example, the physician, will be able to decide exactly which upper and lower limits to use. Alternatively, the range is from about 60 to about 130, preferably, from about 70 to about 120, more preferred, from about 80 to about 110 mg/dL.

Each of the essential ingredients, viz., a therapeutically effective amount of (a) a TF antagonist and (b) Blood glucose regulator have a therapeutically effective interval, namely, the interval of time in which each agent provides benefit for the patient being treated with Thrombotic or Coagulopathic related disease, Inflammatory Disease or Respiratory Disease. The method of the invention may be practiced by separately dosing the patient in any order with a therapeutically effective amount of (a) a TF antagonist and (b) Blood glucose regulator provided that each agent is given within the period of time that that the other agent is therapeutically effective against Thrombotic or Coagulopathic related disease, Inflammatory Disease or Respiratory Disease, or organ failure resulting from these pathologic processes.

The Blood glucose regulator and TF antagonist are preferably administered parenterally to a patient to insure their delivery into the bloodstream in an effective form as fast as possible.

The amount and relative ratio of blood glucose regulator and TF antagonist to be used in the practice of the method of invention is set out in the previous section. It may be appreciated that it may be necessary to make routine variations to the dosage of either agent depending on the age and condition of the patient. The decision to begin the therapy will be based upon the appearance of the clinical manifestations of Thrombotic or Coagulopathic related disease, Inflammatory Disease or Respiratory Disease.

Assays:

Factor VII Biological Activity

The biological activity of factor VIIa in blood clotting derives from its ability to (i) bind to tissue factor (TF) and (ii) catalyze the proteolytic cleavage of factor IX or factor X to produce activated factor IX or X (factor IXa or Xa, respectively).

Biological activity of factor VII polypeptides (“factor VII biological activity”) may be quantified by measuring the ability of a preparation to promote blood clotting using factor VII-deficient plasma and thromboplastin, as described, e.g., in U.S. Pat. No. 5,997,864. In this assay, biological activity is expressed as the reduction in clotting time relative to a control sample and is converted to “factor VII units” by comparison with a pooled human serum standard containing 1 unit/ml factor VII activity. Alternatively, factor VIIa biological activity may be quantified by

(i) Measuring the ability of factor VIIa or a factor VIIa -related polypeptide to produce activated factor X (factor Xa) in a system comprising TF embedded in a lipid membrane and factor X. (Persson et al., J. Biol. Chem. 272:19919-19924, 1997);

(ii) Measuring factor X hydrolysis in an aqueous system (“In Vitro Proteolysis Assay”, see below);

(iii) Measuring the physical binding of factor VIIa or a factor VIIa -related polypeptide to TF using an instrument based on surface plasmon resonance (Persson, FEBS Letts. 413:359-363, 1997); and

(iv) Measuring hydrolysis of a synthetic substrate by factor VIIa and/or a factor VIIa -related polypeptide (“in Vitro Hydrolysis Assay”, see below); and

(v) Measuring generation of thrombin in a TF-independent in vitro system.

In Vitro Hydrolysis Assay

Native (wild-type) factor VIIa and factor VIIa variant (both hereafter referred to as “factor VIIa”) may be assayed for specific activities. They may also be assayed in parallel to directly compare their specific activities. The assay is carried out in a microtiter plate (MaxiSorp, Nunc, Denmark). The chromogenic substrate D-lle-Pro-Arg-p-nitroanilide (S-2288, Chromogenix, Sweden), final concentration 1 mM, is added to factor VIIa (final concentration 100 nM) in 50 mM Hepes, pH 7.4, containing 0.1 M NaCl, 5 mM CaCl₂ and 1 mg/ml bovine serum albumin. The absorbance at 405 nm is measured continuously in a SpectraMaxTm 340 plate reader (Molecular Devices, USA). The absorbance developed during a 20-minute incubation, after subtraction of the absorbance in a blank well containing no enzyme, is used to calculate the ratio between the activities of variant and wild-type factor VIIa:

Ratio=(A_{405 nm} factor VIIa variant)/(A_{405 nm} factor VIIa wild-type).

Based thereon, factor VIIa variants with an activity comparable to or higher than native factor VIIa may be identified, such as, for example, variants where the ratio between the activity of the variant and the activity of native factor VII (wild-type FVII) is around, versus above 1.0.

The activity of factor VIIa or factor VIIa variants may also be measured using a physiological substrate such as factor X, suitably at a concentration of 100-1000 nM, where the factor Xa generated is measured after the addition of a suitable chromogenic substrate (e.g. S-2765). In addition, the activity assay may be run at physiological temperature.

In Vitro Proteolysis Assay

Native (wild-type) factor VIIa and factor VIIa variant (both hereafter referred to as “factor VIIa”) are assayed in parallel to directly compare their specific activities. The assay is carried out in a microtiter plate (MaxiSorp, Nunc, Denmark). factor VIIa (10 nM) and factor X (0.8 microM) in 100 microL 50 mM Hepes, pH 7.4, containing 0.1 M NaCl, 5 mM CaCl2 and 1 mg/ml bovine serum albumin, are incubated for 15 min. factor X cleavage is then stopped by the addition of 50 microL 50 mM Hepes, pH 7.4, containing 0.1 M NaCl, 20 mM EDTA and 1 mg/ml bovine serum albumin. The amount of factor Xa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMaX™ 340 plate reader (Molecular Devices, USA). The absorbance developed during 10 minutes, after subtraction of the absorbance in a blank well containing no FVIIa, is used to calculate the ratio between the proteolytic activities of variant and wild-type factor VIIa:

Ratio=(A405 nm factor VIIa variant)/(A405 nm factor VIIa wild-type).

Based thereon, factor VIIa variants with an activity comparable to or higher than native factor VIIa may be identified, such as, for example, variants where the ratio between the activity of the variant and the activity of native factor VII (wild-type FVII) is around, versus above 1.0.

Inhibition of FVIIa/phospholipids-embedded TF-catalyzed Activation of FX by TF Antagonists—FXa Generation Assay (Assay 1):

In the following example all concentrations are final. Lipidated TF (10 pM), FVIIa (100 pM) and TF antagonist or FFR-rFVIIa (0-50 nM) in HBS/BSA (50 mM hepes, pH 7.4, 150 mM NaCl, 5 mM CaCl2, 1 mg/ml BSA) are incubated 60 min at room temperature before FX (50 nM) is added. The reaction is stopped after another 10 min by addition of ½ volume stopping buffer (50 mM Hepes, pH 7.4, 100 mM NaCl, 20 mM EDTA). The amount of FXa generated is determined by adding substrate S2765 (0.6 mM, Chromogenix, and measuring absorbance at 405 nm continuously for 10 min. IC50 values for TF antagonist inhibition of FVIIa/lipidated TF-mediated activation of FX may be calculated. The IC50 value for FFR-rFVIIa is 51+/−26 pM in this assay.

Inhibition of FVIIa/cell Surface TF-catalyzed Activation of FX by TF Antagonists—FXa Generation Assay (Assay 2):

In the following example all concentrations are final. Monolayers of human lung fibroblasts WI-38 (ATTC No. CCL-75) or human bladder carcinoma cell line J82 (ATTC No. HTB-1) or human keratinocyte cell line CCD 1102KerTr (ATCC no. CRL-2310) constitutively expressing TF are employed as TF source in FVIIa/TF catalyzed activation of FX. Confluent cell monolayers in a 96-well plate are washed one time in buffer A (10 mM Hepes, pH 7.45, 150 mM NaCl, 4 mM KCl, and 11 mM glucose) and one time in buffer B (buffer A supplemented with 1 mg/ml BSA and 5 mM Ca2+). FVIIa (1 nM), FX (135 nM) and varying concentrations of TF antagonist or FFR-rFVIIa in buffer B are simultaneously added to the cells. FXa formation is allowed for 15 min at 37° C. 50-μl aliquots are removed from each well and added to 50 μl stopping buffer (Buffer A supplemented with 10 mM EDTA and 1 mg/ml BSA). The amount of FXa generated is determined by transferring 50 μl of the above mixture to a microtiter plate well and adding 25 μl Chromozym X (final concentration 0.6 mM) to the wells. The absorbance at 405 nm is measured continuously and the initial rates of color development are converted to FXa concentrations using an FXa standard curve. The IC50 value for FFR-rFVIIa is 1.5 nM in this assay.

Inhibition of 1251-FVIIa Binding to Cell Surface TF by TF Antagonists—TF Binding Assay (Assay 3):

In the following example all concentrations are final. Binding studies are employed using the human bladder carcinoma cell line J82 (ATTC No. HTB-1) or the human keratinocyte cell line (CCD1102KerTr ATCC No CRL-2310) or NHEK P166 (Clonetics No. CC-2507) all constitutively expressing TF. Confluent monolayers in 24-well tissue culture plates are washed once with buffer A (10 mM Hepes, pH 7.45, 150 mM NaCl, 4 mM KCl, and 11 mM glucose) supplemented with 5 mM EDTA and then once with buffer A and once with buffer B (buffer A supplemented with 1 mg/ml BSA and 5 mM Ca2+). The monolayers are preincubated 2 min with 100 μl cold buffer B. Varying concentrations of Mabs (or FFR-FVIIa) and radiolabelled FVIIa (0.5 nM 1251-FVIIa) are simultaneously added to the cells (final volume 200 μl). The plates are incubated for 2 hours at 4° C. At the end of the incubation, the unbound material is removed, the cells are washed 4 times with ice-cold buffer B and lysed with 300 μl lysis buffer (200 mM NaOH, 1% SDS and 10 mM EDTA). Radioactivity is measured in a gamma counter (Cobra, Packard Instruments). The binding data are analyzed and curve fitted using GraFit4 (Erithacus Software, Ltd., (U.K.). The IC50 value for FFR-rFVIIa is 4 nM in this assay.

Biosensor Assay (Assay 4):

TF antagonists are tested on the Biacore instrument by passing a standard solution of the TF antagonist over a chip with immobilized TF. This is followed by different concentrations of sTF in 10 mM hepes pH 7.4 containing 150 mM NaCl, 10 mM CaCl2, and 0.0003% polysorbate 20.

Kd's are calculated from the sensorgrams using the integrated Biacore evaluation software.

What is claimed is:

1. A composition comprising a tissue factor antagonist, a blood glucose regulator, and a pharmaceutically acceptable carrier, diluent, and/or excipient.

2. The composition of claim 1, wherein the TF antagonist is a factor VII polypeptide that has a substantially reduced ability to catalyze factor X to factor Xa as compared to wild-type human factor VII.

3. The composition of claim 1, wherein the TF antagonist is a factor VII polypeptide that has been catalytically inactivated in the active site.

4. The composition of claim 3, wherein the TF antagonist is wild-type human factor VII that has been catalytically inactivated in the active site.

5. The composition of claim 3, wherein the factor VII polypeptide is catalytically inactivated in the active site by treatment with a chloromethyl ketone inhibitor independently selected from the group consisting of Phe-Phe-Arg chloromethyl ketone, Phe-Phe-Arg chloromethylketone, D-Phe-Phe-Arg chloromethyl ketone, D-Phe-Phe-Arg chloromethylketone, Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone, D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone and Dansyl-D-Glu-Gly-Arg chloromethylketone.

6. The composition of claim 1, wherein the TF antagonist is an antibody against TF or an antigenic fragment of an antibody against TF.

7. The composition of claim 6, wherein the TF antagonist is a fully human monoclonal antibody or a humanized antibody.

8. The composition of claim 6, wherein the TF antagonist is selected from the group consisting of a Fab fragment; a monovalent fragment consisting of the VL, VH, CL and CH I domains; a F(ab)2 fragment; a F(ab′)2 fragment; a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment; an isolated complementarity determining region (CDR); and a single chain Fv (scFv).

9. The composition of claim 1, wherein the blood glucose regulator is insulin or a salt thereof.

10. The composition of claim 9, wherein the blood glucose regulator is porcine insulin, human insulin, a zinc salt of either thereof, or a protamine salt of either thereof.

11. The composition of claim 1, wherein the blood glucose regulator is an insulin analogue or insulin derivative.

12. The composition of claim 11, wherein the blood glucose regulator is selected from the group consisting of AspB28 human insulin, LysB28-ProB29 human insulin, GlyA21-ArgB31-ArgB32 human insulin, and des-ThrB30 human insulin gamma-LysB29 tetradecanoyl.

13. A method for inducing, promoting, and/or enhancing a physiological response associated with the treatment and/or prevention of a thrombotic or coagulopatic disease, respiratory disease, or inflammatory disease associated with TF in a subject suffering from or at risk of developing such a disease comprising administering a TF antagonist and a blood glucose regulator to the subject in amounts sufficient to detectably induce, promote, and/or enhance the physiological response.

14. The method of claim 13, wherein the TF antagonist and the blood glucose regulator are administered in single-dosage form.

15. The method of claim 13, wherein the method comprises administering a first dosage form comprising a TF antagonist and a second dosage form comprising a blood glucose regulator.

16. The method of claim 13, wherein the subject is suffering from or at risk of developing systemic inflammatory response syndrome, acute lung injury, acute respiratory distress syndrome, disseminated intravascular coagulation, sepsis, CIP, and/or multiple organ failure resulting from any of the preceding pathologic processes.

17. The method of claim 13, wherein the TF antagonist and blood glucose regulator are delivered to the subject by injection.

18. The method of claim 13, wherein the blood glucose regulator is an insulin, a salt of an insulin, an insulin analogue, or an insulin derivative.

19. The method of claim 13, wherein the TF antagonist is a factor VII polypeptide that has been catalytically inactivated in the active site.

20. The method of claim 13, wherein the TF antagonist is a fully human monoclonal antibody or a humanized antibody against TF.


SEQUENCE LISTING (1 sequence)

SEQ ID NO: 1 - protein, 406 aa, human coagulation Factor VII
  MISC_FEATURE (1)..(406): Xaa=4-carboxyglutamic acid (gamma- carboxyglutamate)
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP